(19) United States
(12) Patent Application Publication
(10) Pub. No.: US 2019/0240247 A1
(43) Pub. Date: Aug. 8, 2019

(54) PHARMACEUTICAL FORMULATIONS OF REGADENOSON

(71) Applicant: LEIUTIS PHARMACEUTICALS PVT. LTD., Hyderabad (IN)

(72) Inventors: Kocherlakota CHANDRASHEKHAR, Secunderabad (IN); Banda NAGARAJU, Hyderabad (IN)

(73) Assignee: LEIUTIS PHARMACEUTICALS PVT. LTD., Hyderabad (IN)

(21) Appl. No.: 16/329,864

(22) PCT Filed: Aug. 31, 2017
(86) PCT No.: PCT/IB2017/055239

§ 371 Date: Mar. 1, 2019

(30) Foreign Application Priority Data
Sep. 1, 2016 (IN) 201641029943

(51) Int. Cl. A61K 31/7076; A61K 9/00; A61K 47/40; A61K 47/10

(52) U.S. Cl. CPC A61K 31/7076; A61K 47/10; A61K 47/40; A61K 9/0019

(57) ABSTRACT

The present invention relates to parenteral pharmaceutical formulation of Regadenoson comprising of Regadenoson, one or more cyclodextrins and pharmaceutically acceptable excipients.

### BACKGROUND OF THE INVENTION

Regadenoson is chemically described as 2-[4-[(methylamino) carbonyl]-1H-pyrazol-1-yl]-adenosine. Regadenoson is a selective A_{2A} adenosine receptor agonist that is a coronary vasodilator and has the following structure:

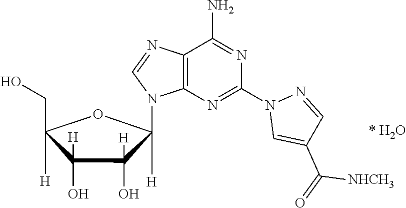

Regadenoson is used as a pharmacologic stress agent indicated for radionuclide myocardial perfusion imaging (MPI) in patients unable to undergo adequate exercise stress. MPI is a diagnostic technique useful for the detection and characterization of coronary artery disease.

Regadenoson and related compounds as well as methods for their manufacture and use in cardiac perfusion imaging are disclosed in U.S. Pat. Nos. 6,403,567; 6,642,210; 6,214,807; 7,655,636; 8,071,566; 8,106,029 and 6,770,634 as well as published in U.S. patent application no. 2002/012946.

U.S patent application No. 2013/004426 to Ajit et al., discloses a composition comprising (a) an adenosine derivative, which is methyl trans-4-[3-[6-amino-9-(N-ethyl-3-D-ribofuranosyluronamide)-9H-purin-2-yl]prop-2-ynyl]cyclohexane carboxylate and (b) a solvent consisting essentially of water and hydroxypropyl 3 cyclodextrin.

U.S. Pat. No. 8,133,879 to Belardinelli et al., describes pharmaceutical compositions comprising a) Regadenoson b) liquid carriers such as water c) phosphate buffer d) EDTA and e) propylene glycol.

U.S. Pat. No. 8,106,029 to Gordi et al., describes method of producing coronary vasodilation by administering pharmaceutical compositions of Regadenoson as a single intravenous bolus dose.

U.S. Pat. No. 7,655,636 to Gordi et al., describes method of producing coronary vasodilation with little peripheral vasodilation comprising administering to a human a single dose of a pharmaceutical composition comprising Regadenoson and at least one pharmaceutical excipient.

PCT Publication No. WO2014/083580 to Chandrashekhar et al., describes pharmaceutical composition of Regadenoson. Cyclodextrins are disclosed in the said patent application as one of the optional excipients. However, there is no special teaching of how to practice the invention with cyclodextrins.

Commercially, Regadenoson is available as solution for intravenous injection under the brand name Lexiscan® in the United States. Lexiscan is supplied as a sterile, preservative-free solution containing 0.08 mg/mL Regadenoson, available in single-use 5 mL pre-filled plastic Ansyr® syringes. Each 1 mL in the 5-mL pre-filled syringe contains 0.084 mg of Regadenoson monohydrate, corresponding to 0.08 mg Regadenoson on an anhydrous basis, 10.9 mg dibasic sodium phosphate dihydrate or 8.7 mg dibasic sodium phosphate anhydrous, 5.4 mg monobasic sodium phosphate monohydrate, 150 mg propylene glycol, 1 mg edetate disodium dihydrate and water for injection, with pH between 6.3 and 7.7.

Regadenoson formulations include co-solvents due to low aqueous solubility of Regadenoson. Propylene glycol was identified as an appropriate co-solvent to formulate Lexiscan. Even though propylene glycol is considered harmless, in high concentrations, it increases the formulation osmolality and may cause undesirable side effects. Hence there is a need to develop improved formulations of Regadenoson.

### SUMMARY OF THE INVENTION

One aspect of the invention is to provide a parenteral pharmaceutical formulation of Regadenoson comprising of Regadenoson, one or more cyclodextrins and other pharmaceutically acceptable excipients, wherein the formulation is free of propylene glycol.

Another aspect of the present invention provides a parenteral pharmaceutical formulation of Regadenoson comprising of Regadenoson, one or more cyclodextrins, one or more solvents and optionally other pharmaceutically acceptable excipients, wherein the formulation is free of propylene glycol.

Yet another aspect of the invention provides a parenteral pharmaceutical formulation of Regadenoson, wherein the ratio of Regadenoson to the cyclodextrin(s) is at least about 1:0.05 by weight.

### DETAILED DESCRIPTION OF THE INVENTION

In the context of this invention, the term “Regadenoson” includes its pharmaceutically acceptable salts, solvates or hydrates thereof.

The term “free of” is intended to mean formulations that have less than 5%, or more specifically less than 2% of the said component.

The term “about”, as used herein, refers to any value which lies within the range defined by a variation of up to +10% of the value.

Cyclodextrins are often used in pharmaceutical formulations because of their ability to increase the solubility and stability. Cyclodextrins are a group of cyclic oligomers containing α-D-glucopyranose units linked with α-1-4 bonds. The cyclodextrin can be a natural cyclodextrin or a modified cyclodextrin or a mixture. There are three well known naturally occurring cyclodextrins—α, β and γ, which are composed of six, seven or eight glucopyranose units, respectively. Modified cyclodextrins can include, for example, hydroxy propyl β cyclodextrin, sulfoalkyl-cyclodextrin, methylated cyclodextrin, ethylated cyclodextrin, sulfoalkyl-ether β cyclodextrin and mixtures.

The parenteral formulations of Regadenoson conventionally use propyleneglycol which is not a preferred excipient particularly when used in high quantities. The invention relates to parenteral formulations of Regadenoson free of propylene glycol and comprising one or more cyclodextrins, which is not taught in the prior art.

One embodiment of the invention relates to a parenteral pharmaceutical formulation of Regadenoson comprising
a) Regadenoson
b) one or more cyclodextrins
c) one or more solvents and
d) optionally other pharmaceutically acceptable excipients,
wherein the formulation is free of propylene glycol.

Another embodiment of the invention relates to a parenteral pharmaceutical formulation of Regadenoson comprising
a) Regadenoson
b) one or more cyclodextrins selected from the group comprising hydroxypropyl β-cyclodextrins (HPCD), methyl-and-ethyl-β-cyclodextrin, sulfoalkylether-substituted β-cyclodextrin, sulfobutylether-β-cyclodextrin (SBECD)
c) one or more solvents selected from ethanol, glycerine, polyethylene glycol, water and
d) optionally other pharmaceutically acceptable excipients,
wherein the formulation is free of propylene glycol.

Yet another embodiment of the invention relates to a parenteral pharmaceutical formulation of Regadenoson, wherein the ratio of Regadenoson to the cyclodextrin is atleast about 1:0.05 by weight. Preferably the ratio of Regadenoson to the cyclodextrin is atleast about 1:100, more preferably atleast about 1:300, more preferably atleast 1:1500 by weight.

Suitable cyclodextrins include but are not limited to a, 3 and γ-cyclodextrin and cyclodextrins modified with alkyl, hydroxyalkyl, dialkyl and sulfoalkyl-ether modified cyclodextrins such as methyl or hydroxypropyl β-cyclodextrins (HPCD), methyl-and-ethyl-β-cyclodextrin, sulfoalkylether-substituted β-cyclodextrin, sulfobutylether-β-cyclodextrin (SBECD) and the like. Preferably sulfobutylether-β-cyclodextrin (SBECD) and HPCD are used.

Solvents that may be used in the formulation include ethanol, glycerine, polyethylene glycols and water. Preferred solvent is water. The quantity of solvent(s) may range up to 99% based on total weight of the formulation.

The formulation of the present invention may comprise other pharmaceutically acceptable excipients selected from group comprising buffering agents, solvents,chelating agents, pH adjusting agents, anti-oxidants and the like.

Suitable buffering agents include citrate, glutamate, dicarboxylic acid, bicarbonate, tartrate, benzoate, lactate, gluconate, acetate, borate, meglumine, TRIS buffer, amino acids such as arginine, alanine, histidine, glycine, lysine and the like.

Suitable pH adjusting agents include sodium hydroxide, sodium carbonate, sodium bicarbonate, potassium hydroxide, hydrochloric acid, citric acid, sulfuric acid, and the like.

The pharmaceutical compositions of the present invention may also contain one or more anti-oxidants such as butylated hydroxyanisole, butylated hydroxyl toluene, tocopherol, monothioglycerol, ascorbic acid and L-cysteine.

Suitable chelating agents include, DOTA (1,4,7,10-tetraazacyclododecane-1,4,7,10-tetraacetic acid), DTPA (diethylenetriamine pentaacetic acid), EGTA (ethylene glycol-bis(3-aminoethyl ether)-N,N,N′,N′-tetraacetic acid), HEDTA (N (hydroxy ethyl) ethylenediaminetriacetic acid), EDTA (Ethylenediamine tetraacetic acid), DOTRP (tetraethyleneglycol-1,5,9-triazacyclododecane-N,N′,N″,-tris(methylenephosphonic acid) its salts and analogues thereof.

A preferred embodiment of the invention comprises
a) Regadenoson
b) sulfobutylether-β-cyclodextrin (SBECD) or hydroxypropyl 3-cyclodextrins (HPCD)
c) buffering agent
d) ethylenediamine tetraacetic acid (EDTA) or 1,4,7,10-tetraazacyclododecan-1,4,7,10-tetraacetic acid (DOTA) or diethylenetriamine pentaacetic acid (DTPA) and their salts
e) water and
f) optionally other pharmaceutically acceptable excipients selected from pH adjusting agents, buffering agents and anti-oxidants, wherein the formulation is free of propylene glycol.

Regadenoson formulations prepared according to the invention were tested for stability at 25° C./60% RH, 30° C./65% RH and 40° C./75% RH for a period of 3 months. The stability data of the invention formulation is summarized in table 1.

TABLE 1
Stability data of the invention formulation prepared according
to example 1.

| | 25° C./ | 30° C./ | |
|---|---|---|---|
| | 60% RH | 65% RH | 40° C./75% RH |
| Parameters | 3 M | 3 M | 3 M |
| pH | 7.21 | 7.23 | 6.99 |
| Osmolality (mOsmol/kg) | 275 | 275 | 274 |
| Assay (%) | 99.8 | 99.3 | 98.2 |
| Total impurities | 0.21 | 0.23 | 0.73 |

From the above results, it is evident that the formulations prepared according to the invention are stable.

The following examples further describe certain specific aspects and embodiments of the present invention. It is to be understood that the examples are given by way of illustration only.

### Example 1

| S. No | Ingredients | Qty per mL | |
|---|---|---|---|
| 1 | Regadenoson | 0.08 | mg |
| 2 | Glycine | 20 | mg |
| 3 | Sulfobutylether β-cyclodextrin (SBECD) | 0.012 | mg |
| 4 | DOTA | 0.50 | mg |
| 5 | Monothioglycerol | 0.15 | mg |
| 6 | Sodium Hydroxide | Q.S. to adjust pH | |
| 7 | Hydrochloric acid | Q.S. to adjust pH | |
| 8 | Water for injection | Q.s to 1 | mL |

Q.S: Quantity sufficient

Manufacturing Procedure:

Water for injection was taken in a vessel and SBECD was added. The solution was heated to 50±5° C. and Regadenoson was added and stirred till a clear solution was obtained. The solution was then cooled to room temperature. Glycine was added followed by the addition of DOTA and monothioglycerol and stirred. pH of the solution was adjusted to 7.0±0.1 using sodium hydroxide or hydrochloric acid solution.

### Example 2

| S. No | Ingredients | Qty per mL | |
|---|---|---|---|
| 1 | Regadenoson | 0.08 | mg |
| 2 | Histidine | 19 | mg |
| 3 | Hydroxypropyl β-cyclodextrins (HPCD) | 0.02 | |
| 4 | Diethylenetriamine pentaacetic acid (DTPA) | 0.5 | mg |
| 5 | Monothioglycerol | 0.15 | mg |
| 6 | Sodium Hydroxide | Q.S. to adjust pH | |
| 7 | Hydrochloric acid | Q.S. to adjust pH | |
| 8 | Water for injection | Q.s to 1 | mL |

Manufacturing Procedure:

Water for injection was taken in a vessel and HPCD was added. The solution was heated to 50±5° C. and Regadenoson was added and stirred till a clear solution was obtained. The solution was then cooled to room temperature. Histidine was added followed by the addition of DTPA and monothioglycerol and stirred. pH of the solution was adjusted using sodium hydroxide or hydrochloric acid solution.

### Example 3

| S. No | Ingredients | Qty per mL | |
|---|---|---|---|
| 1 | Regadenoson | 0.08 | mg |
| 2 | Tris | 3 | mg |
| 3 | Sulfobutylether β-cyclodextrin (SBECD) | 0.016 | mg |
| 4 | Diethylenetriamine pentaacetic acid (DTPA) | 0.50 | mg |
| 5 | Monothioglycerol | 0.15 | mg |
| 6 | Sodium Hydroxide | Q.S. to adjust pH | |
| 7 | Hydrochloric acid | Q.S. to adjust pH | |
| 8 | Water for injection | Q.s to 1 | mL |

Manufacturing Procedure

Water for injection was taken in a vessel and SBECD was added. The solution was heated to 50±5° C. and Regadenoson was added and stirred till a clear solution was obtained. The solution was then cooled to room temperature. Tris buffer was added followed by the addition of DTPA and monothioglycerol and stirred. pH of the solution was adjusted using sodium hydroxide or hydrochloric acid solution.

### Example 4

| S. No | Ingredients | Qty per mL | |
|---|---|---|---|
| 1 | Regadenoson | 0.08 | mg |
| 2 | Glycine | 20 | mg |
| 3 | Sulfobutylether-β-cyclodextrin (SBECD) | 100 | mg |
| 4 | DOTA | 0.50 | mg |
| 5 | Monothioglycerol | 0.15 | mg |
| 6 | Sodium Hydroxide | Q.S. to adjust pH | |
| 7 | Hydrochloric acid | Q.S. to adjust pH | |
| 8 | Water for injection | Q.s to 1 | mL |

Manufacturing Procedure

Water for injection was taken in a vessel and SBECD was added. The solution was heated to 50±5° C. and Regadenoson was added and stirred till a clear solution was obtained. Glycine was added followed by the addition of DOTA and monothioglycerol and stirred. pH of the solution was adjusted using sodium hydroxide or hydrochloric acid solution. The solution was cooled to room temperature, filtered and filled into suitable containers.

### Example 5

| S. No | Ingredients | Qty per mL | |
|---|---|---|---|
| 1 | Regadenoson | 0.08 | mg |
| 2 | Glycine | 20 | mg |
| 3 | Sulfobutylether-β-cyclodextrin (SBECD) | 100 | mg |
| 4 | EDTA | 0.50 | mg |
| 5 | Monothioglycerol | 0.15 | mg |
| 6 | Sodium Hydroxide | Q.S. to adjust pH | |
| 7 | Hydrochloric acid | Q.S. to adjust pH | |
| 8 | Water for injection | Q.s to 1 | mL |

Manufacturing Procedure

Water for injection was taken in a vessel and SBECD was added. The solution was heated to 50±5° C. and Regadenoson was added and stirred till a clear solution was obtained. Glycine was added followed by the addition of EDTA and monothioglycerol and stirred. pH of the solution was adjusted using sodium hydroxide or hydrochloric acid solution. The solution was cooled to room temperature, filtered and filled into a suitable container.

### Example 6

| S. No | Ingredients | Qty per mL | |
|---|---|---|---|
| 1 | Regadenoson | 0.08 | mg |
| 2 | Glycine | 20 | mg |
| 3 | Sulfobutylether-β-cyclodextrin (SBECD) | 50 | mg |
| 4 | DOTA | 0.50 | mg |
| 5 | Monothioglycerol | 0.15 | mg |
| 6 | Sodium Hydroxide | Q.S. to adjust pH | |
| 7 | Hydrochloric acid | Q.S. to adjust pH | |
| 8 | Water for injection | Q.s to 1 | mL |

Manufacturing Procedure

Water for injection was taken in a vessel and SBECD was added. The solution was heated to 50±5° C. and Regadenoson was added and stirred till a clear solution was obtained. Glycine was added followed by the addition of DOTA and monothioglycerol and stirred. pH of the solution was adjusted using sodium hydroxide or hydrochloric acid solution. The solution was cooled to room temperature, filtered and filled into a suitable container.

We claim:

1. A parenteral pharmaceutical formulation of Regadenoson comprising:
(i) Regadenoson or a pharmaceutically acceptable salt, solvates or hydrates thereof
(ii) one or more cyclodextrins
(iii) one or more solvents and
(iv) optionally other pharmaceutically acceptable excipients,
wherein the formulation is free of propylene glycol.

2. The parenteral pharmaceutical formulation of claim 1, wherein the cyclodextrins are selected from, methyl or hydroxypropyl β-cyclodextrins (HPCD), methyl and ethyl β-cyclodextrin, sulfoalkylether-substituted β-cyclodextrin, sulfobutylether-β-cyclodextrin (SBECD).

3. The parenteral pharmaceutical formulation of claim 1, wherein the ratio of Regadenoson to the cyclodextrin(s) is at least about 1:0.05 by weight.

4. The parenteral pharmaceutical formulation of claim 1, wherein the solvents are selected from ethanol, glycerine, polyethylene glycol and water.

5. The parenteral pharmaceutical formulation of claim 1, wherein the other pharmaceutically acceptable excipients can be selected from buffering agents, pH adjusting agents and anti-oxidants.
